(19) United States
(12) Patent Application Publication
(10) Pub. No.: US 2025/0040841 A1
(43) Pub. Date: Feb. 6, 2025

(54) METHOD FOR DETERMINING THE CONCENTRATION OF A SUBSTANCE AND DETECTOR ARRANGMENT

(71) Applicant: ams-OSRAM International GmbH, Regensburg (DE)

(72) Inventors: Klaus FLOCK, Regensburg (DE); Alexander PREBECK, München (DE)

(21) Appl. No.: 18/717,415

(22) PCT Filed: Dec. 7, 2022
(86) PCT No.: PCT/EP2022/084824

§ 371 Date: Jun. 6, 2024

(30) Foreign Application Priority Data
Dec. 7, 2021 (DE) 10 2021 132 135.2

(51) Int. Cl. A61B 5/1455; A61B 5/145

(52) U.S. Cl. CPC A61B 5/1455; A61B 5/14532

(57) ABSTRACT

A method for determining a substance concentration in a particle-containing liquid, in particular of glucose in blood, wherein a refractive index of the liquid is dependent on a concentration of the substance dissolved therein, includes emitting a measuring light beam of at least one wavelength onto a sample containing the liquid. The method also includes detecting a first light component scattered by scattering on the particles contained in the liquid at a first angle, which corresponds in particular to a forward scattering of the light component. The method further includes detecting a second light component scattered at a second angle by the particles contained in the liquid. The method additionally includes determining a concentration or a proportion of the substance in the liquid from the detected first and second light components, in particular by forming a ratio from the detected first and second light components.

The present application claims the priority of the German application DE 10 2021 132 135.2 of Dec. 7, 2021, the disclosure of which is hereby incorporated by reference in its entirety.

The present invention relates to a method for determining a substance concentration in a particle-containing liquid, in particular of glucose in blood, wherein a refractive index of the liquid is dependent on a concentration of the substance dissolved therein.

### BACKGROUND

The current standard for blood glucose monitoring often uses an invasive technique in which a small amount of blood is drawn and a subsequent electrochemical analysis is performed with a handheld device. This method is not suitable for continuous monitoring, as the finger must be pricked for each measurement, in order to take a fresh blood sample. A more recently developed technology works with a button that sits on the skin and measures interstitial fluid in parts of the fatty tissue of the subcutis with a small, needle-like sensor. However, the needle permanently penetrates the skin.

In addition to these invasive methods, there are also non-invasive methods based on optical IR measurements or Raman spectroscopy, among others. While in the first case, a suitable choice of emitter and detector leads to difficulties, the approach based on Raman spectroscopy is a challenge due to the very poor signal-to-noise ratio.

In this respect, there is a need for a method that can detect a substance in a liquid in a simpler way and continuously.

### SUMMARY OF THE INVENTION

This need is met by the objects of the independent patent claims. Further additions and embodiments are shown in the dependent claims.

The inventors have recognized that there is an angular dependence between a scattered light component and a concentration of the substance in a liquid. The latter is in turn based on a change in a refractive index as a function of the concentration of the substance in the liquid. The proposed method utilizes this dependence to obtain a relative measurement between two light components that have been scattered by particles in the liquid, where the light component is a scattered light component.

To illustrate the basic property, it is helpful to think of the measured light signal as a wave that moves in forward direction if there is no index contrast between the liquid and the objects in it. The objects on which the light can scatter would be completely invisible in this environment, i.e. with the same refractive indices between the liquid and the object. However, if the refractive indices differ from each other, an interaction of the light with the matter can be observed, e.g. by some of the light being scattered. This interaction is complicated and depends not only on the refractive indices but also on the shape and size of the particles. Surprisingly, however, there is an angular dependence, i.e. the light scattered by a particle in a liquid is not evenly distributed, but comprises a preferred direction. The angular dependence is in turn partly characteristic of the refractive indices or a change thereof and thus of the concentration of a substance in the liquid.

Based on this finding, the inventors have developed a method for non-invasive tracking of blood glucose levels by evaluating the optical return signal, which essentially propagates in a forward direction relative to a reference direction. The second light component is measured in the reference direction. The invention also makes use of the fact that the distribution of the scattered light is influenced, among other things, by a scattering similar to Mie scattering, since the particles contained in the liquid are of the same order of magnitude as a wavelength of a measuring light beam.

In some aspects, a method for determining a concentration of a substance present in a liquid is proposed. In addition to this substance, the liquid also comprises particles on which a light beam can be scattered. The refractive index of the liquid or also the difference in the refractive indices between the liquid with the substance dissolved therein and the particles is determined in which, in a first step, a measuring light beam of at least one wavelength strikes a sample containing the liquid and is scattered in or on it. A portion of light scattered at a first angle, in particular at a first angle corresponding to the measuring light beam in the forward direction, is then detected. The scattered light detected in this way is based at least partially or, depending on the measuring angle and wavelength of the light, on angle-dependent scattering, e.g. on scattering comparable to Mie scattering. Scattering components due to Rayleigh scattering, on the other hand, are of lesser importance due to the size of the particles.

A second light component is also detected at a second angle, which is scattered on or in the liquid. An angle dependency and thus a concentration or proportion of the substance in the liquid can be determined from the two detected scattered light components. In particular, such a determination can be made by evaluating a ratio of the detected first and second light components.

The inventors thus propose a method which utilizes the angular dependence in the scattering comparable to Mie scattering at different refractive indices between liquid and particles in the same or differences thereof, whereby one of the refractive indices depends on the concentration of the substance to be determined.

The sample is illuminated at two different angles or a scattered light component is detected at two different angles. Depending on the application, various combinations of these can be selected in order to obtain a good signal/noise result. Although scattering comparable to Mie scattering comprises only a slight wavelength dependence, measurement at different wavelengths is possible and, depending on the application, also useful in order to compensate for other effects in the sample or the particles. This may seem particularly useful if a surface of the sample comprises a different characteristic and the measurement is to take place below the sample, so that the measuring light should penetrate the surface with as little absorption or other effects as possible.

The proposed method is particularly suitable for determining a glucose concentration in blood, as the size of the red blood cells moves in the range of the wavelength of the measuring light. At the same time, other parameters can also be measured so that the method can be used both for determining glucose alone and in combination with other methods. Examples of this would be pulse measurement, blood pressure measurement and the like. Other substances, such as alcohol or lactate, can also be detected in the blood.

As mentioned above, there are different variations for sending light onto the sample at different angles and for detecting the scattered light, whereby at least one light scattered in the forward direction and at least a second light scattered in a different direction are detected. It should be mentioned at this point that light can generally be scattered in all spatial directions. Therefore, the term “scattered light beam” refers to a portion of light detected by a detector. The term “in the forward direction” is thus understood in some aspects to mean that an angle between the measuring light beam and the first light component generated by scattering on the particles contained in the liquid and detected by a detector is more than 90° and in particular more than 120°.

Accordingly, in some aspects, a first light component is detected in the forward direction, whereby this light component is caused by scattering comparable to Mie scattering. In other words, the measuring light beam falls flat on the surface of the sample, i.e. at an angle smaller than 40° and in particular smaller than 30° (or at a correspondingly large angle to the normal). A detector for measuring the scattered light is positioned in such a way that it receives light scattered at a flat angle in the forward direction.

In another aspect, the above-mentioned first angle relative to a normal to a surface of the sample is greater than 45° and in particular greater than 60°. Further or alternatively, the second angle, i.e. the angle of the second light component, also referred to as the reference component, may comprise an angle relative to a normal to a surface of the sample of less than 35° and in particular less than 20°. In a related alternative, the second angle relative to the normal to the surface of the sample may be greater than 45° and also greater than 60°, but the second light component is then formed by backscattered light, i.e. backscattered light with an angle greater than 45° or even greater than 60° is detected.

Some other aspects deal with the measuring light beam, i.e. the incident light. In some aspects, the measuring light beam comprises an angle of incidence with respect to a normal to a surface of the sample that is greater than 45° and, in particular, greater than 60°. In other words, the measuring light beam is irradiated onto the sample at a flat angle. While the first light component is detected by forward-scattered light, an angle between the measuring light beam and the detected scattered second light component can be less than 60° and in particular less than less than 30°. The second light component therefore primarily contains backscattered light, which is detected by a detector and fed for further evaluation.

In some aspects, a distinction is made between a measurement light beam and a reference light beam. While the measurement light beam is used to form and subsequently detect forward scattered light, a backscattered light generated by the reference light beam is detected as a second light component. In these cases, either differently positioned emitters can be used so that their generated light beams fall on the sample at different angles but are detected by a single detector.

Thus, in some aspects, the step of detecting a second light portion scattered by the particles contained in the liquid comprises emitting a reference light beam of at least one wavelength onto the sample. A light scattered by the sample is detected at a second angle as the second light component. The reference light beam can be irradiated at a fourth angle with respect to a normal to a surface of the sample, which is less than 45° and in particular less than 30°.

The reference light beam and measuring light beam can be emitted at different times. This prevents the two light beams from influencing each other. Alternatively, different wavelengths can also be used, provided that the detector for detecting the first and/or second light component operates in a wavelength-specific manner.

In an alternative embodiment, a single emitter is used whose light scattered by the sample is detected by several detectors at different angles. In this case, a measuring light beam is generated and light scattered in the forward direction is detected as the first light component. Similarly, a light component scattered at a different angle is detected as the second light component.

In some aspects, the reference light beam and the measurement light beam comprise the same wavelength. In this context, however, it is possible that the two measurement beams comprise several different wavelengths, i.e. are composed of light with different wavelengths. Accordingly, the measurements for the first and second light components can either be carried out simultaneously with light of different wavelengths or with light of the same wavelength but possibly at different times. It is also possible to carry out several measurements with measuring light beams and/or reference light beams of different wavelengths in order to compensate for absorption or other effects in the sample.

A further aspect relates to the position of the incident light beam on the sample surface. In some aspects, it is provided that the measurement light beam and the reference light beam impinge on the same location on the sample surface. In another aspect, the point of incidence of the measurement light beam may be spaced from a location from which backscattered light is detected. This is advantageous if scattered light due to reflection on the surface is not to be detected. It is also possible in this way to receive scattered light that has traveled a greater distance, possibly coming from deeper layers and thus interacting with the liquid and particles to be examined.

A further aspect relates to a measuring arrangement or a sensor for determining a substance concentration in a liquid, and in particular of glucose in sugar.

Such a sensor comprises a housing with an outlet window and an optional inlet window. The exit window and entrance window can be optically separated from each other in order to further reduce crosstalk during a measurement. The optoelectronic sensor comprises at least one emitter unit, which is arranged below the exit window and is designed to emit light from the exit window onto the sample at at least a first angle. In addition, a photodetector unit is provided, which is arranged under the entrance window.

The photodetector unit is designed to detect light scattered by the sample and incident at at least a first angle.

According to the invention, the sensor is designed to detect a first light component in response to a first emitted light and a second light component in response to a second emitted light. In this case, an angle between the first light component and the first emitted light differs by at least 60° and in particular by at least 90° or even at least 110° from an angle between the second light component and the second emitted light.

In other words, the angle between the first light portion and the corresponding light falling on the sample is significantly greater than the angle between the second light portion and the corresponding light falling on the sample. In this context, however, it can still be said that the angle between the first light component and the first emitted light consists of a forward-scattered light component from the first emitted light and the angle between the second light component and the second emitted light consists of a correspondingly backward-scattered light.

In an additional aspect, an evaluation unit is then provided, which is coupled to the at least one photodetector unit. By means of the evaluation unit, a substance concentration in the sample can be determined from a ratio of the detected first and second light components.

The proposed optoelectronic sensor utilizes the scattering of scattering particles in a sample, especially in a liquid sample, and is capable of detecting the forward scattered light generated by the scattering as well as the backward scattered light. It has been found that the scattering is strongly angle-dependent and exhibits some properties that are also characteristic of Mie scattering (less so for Rayleigh scattering), so that in the following this scattering and the term Mie scattering are used synonymously.

The strong angular dependence caused by the scattering allows conclusions to be drawn about the refractive index and, in particular, about a change in the refractive indices in the sample.

Measurements carried out at different times make it possible to determine a change in the concentration of the substance, which in turn causes a change in the refractive index, which can be determined by measuring the Mie scattering. The different light components can be determined either by differently emitted light beams or from a common light beam acting on the sample.

Accordingly, in one embodiment, the optoelectronic sensor comprises at least one measuring emitter which is designed to emit light at a first predetermined angle from the exit window onto the sample. Optionally, a reference emitter can now be provided, which is designed to emit light at a second predetermined angle from the exit window onto the sample. The second predetermined angle is significantly smaller than the first predetermined angle compared to a normal angle to the sample surface. In other words, the measurement emitter is used to emit light from the exit window onto the sample at a particularly flat angle, while the reference emitter in turn emits light onto the sample from as steep an angle as possible. In this context, a flat angle is understood to be a predetermined angle relative to a normal to a surface of the sample that is greater than 45° and in particular greater than 55° or 60° and in particular greater than 65°. In contrast, the second predetermined angle comprises an angle relative to a normal to a surface of the sample which is smaller than 35° and in particular smaller than 20° or smaller than 15°.

In this configuration, the sensor generates two light beams that strike the sample at different angles.

In another aspect, the photodetector unit again comprises a detector adapted to detect light scattered by the sample and incident at at least a first predetermined angle. An optional reference detector can also be provided on this side, which is designed to detect light scattered by the sample and incident at at least a second predetermined angle. Here too, the second predetermined angle is not smaller than the first predetermined angle relative to a normal to the sample surface. Accordingly, in some aspects, the photodetector unit is thus configured to detect scattered light from the sample at two different angles. This again corresponds to a forward scattered light component or a backward scattered light component of a scattering of the sample which comprises at least partially components of a scattering similar to Mie scattering.

The first predetermined angle is also greater than 45° and in particular greater than 55° or 60° relative to a normal to a surface of the sample. The second angle generates a backscattered light component and has a relatively small value compared to a normal to a program surface, in particular less than 35° or less than 20°.

Additional measures may also be provided to improve a signal-to-noise ratio. In some aspects, the electronic sensor comprises an optical barrier for this purpose, which is arranged in the housing and, in particular, extends from the exit window towards the bottom of the housing. The optical barrier is arranged between the emitter unit and the photodetector unit. In this context, several photodetector units or even several emitter units are possible, each of which detects or emits light of different wavelengths. In this way, wavelength-dependent absorption properties of the sample can be compensated for, so that the signal-to-noise ratio and thus also the signal quality is further improved.

A further aspect relates to the embodiment with an optical system, which is connected downstream or upstream of the individual emitters of the emitter unit or the photodetectors of the photodetector unit. On the one hand, the optical system can serve to deflect the light emitted by the emitter unit and to image it onto a specific focal point on the sample surface. Accordingly, the optical system of the photodetector unit is designed to collect the light at the desired angle and direct it onto the photosensor of the detector unit.

In certain applications, it is expedient to set the focal point generated by the emitter unit on the sample at a distance from a point from which outgoing light can be detected by the photodetector unit at the at least one first angle. This is particularly useful if stray light reflected directly from the sample surface is not to be detected. Apertures or other optical barriers can also be provided for this purpose. In fact, such a structure allows the light emitted by the emitter unit to penetrate into the sample and only be scattered at a certain depth by scattering in the direction of the photodetector unit.

In this respect, the portion entering the detector has traveled a greater distance or comes from a greater depth, so that interaction with the liquid and the particles it contains is greatly increased. This makes it possible to determine a substance concentration within a certain depth of the sample.

This aspect is particularly useful in applications in which a glucose concentration of blood from human tissue is to be detected, as blood is located at a certain depth below the surface of the skin. By selecting the distance between the respective focal points, it may be possible to measure the concentration of the substance within the sample in a depth-dependent manner. It has been found that good results can be achieved in certain applications if the distance between the focal point and the point from which the outgoing light can be detected by the photodetector unit is in the range between 1 mm and 6 mm and in particular in the range between 2 mm and 4 mm. There is also a correlation between the wavelength and the distance. Initial results indicate that the correlation between the measurement results and the substance concentration increases if the distance is also slightly smaller at shorter wavelengths than at longer wavelengths.

### BRIEF DESCRIPTION OF THE DRAWING

Further aspects and embodiments according to the proposed principle will become apparent with reference to the various embodiments and examples described in detail in connection with the accompanying drawings.

FIG. 1 shows a top view and a cross-sectional view of a version of an optoelectronic sensor for carrying out the method according to the proposed principle;

FIG. 2 shows a schematic diagram of the optoelectronic sensor and a first measurement step for carrying out the process according to the proposed principle;

FIG. 3 shows a schematic diagram of the optoelectronic sensor and a second measurement step for carrying out the process according to the proposed principle;

FIG. 4 schematically shows a second version of an optoelectronic sensor which is suitable for carrying out the method according to the proposed principle;

FIG. 5 schematically illustrates a third version of an optoelectronic sensor suitable for carrying out the method according to the proposed principle;

FIGS. 6 and 7 each show a signal-time diagram illustrating a time course of the blood glucose concentration measured using two different methods.

### DETAILED DESCRIPTION

The following embodiments and examples show various aspects and their combinations according to the proposed principle. The embodiments and examples are not always to scale. Likewise, various elements may be shown enlarged or reduced in size in order to emphasize individual aspects. It is understood that the individual aspects and features of the embodiments and examples shown in the figures can be readily combined with each other without affecting the principle of the invention. Some aspects comprise a regular structure or shape. It should be noted that slight deviations from the ideal shape may occur in practice without, however, contradicting the inventive concept.

In addition, the individual figures, features and aspects are not necessarily shown in the correct size, and the proportions between the individual elements are not necessarily correct. Some aspects and features are emphasized by enlarging them. However, terms such as “above”, “above”, “below”, “below”, “larger”, “smaller” and the like are shown correctly in relation to the elements in the figures. Thus, it is possible to derive such relationships between the elements based on the figures. However, the proposed principle is not limited to this, but various optoelectronic components with different sizes and also functionality can be used in the invention. In the embodiments, elements with the same or similar effects are shown with the same reference signs.

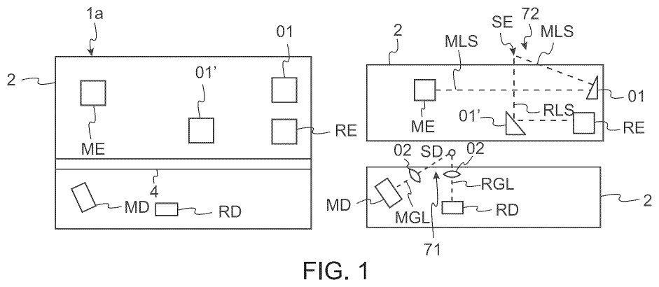

FIG. 1 shows in the left-hand illustration a detector arrangement according to the proposed principle, as it is suitable for carrying out the proposed method. The right-hand illustration shows a section through the two sides of the detector arrangement. The detector arrangement comprises a housing 2, which is separated into two areas by an optical barrier 4. The optical barrier 4 extends from the bottom of the housing 2 to a top side, which comprises an exit window 72 and an entry window 71 slightly offset from it. The corresponding inlet and outlet windows are spaced apart and separated by the optical barrier 4. The optical barrier 4 prevents crosstalk from light emitted through the center to the detectors during a measurement and thus improves the signal-to-noise ratio.

The emitter area comprises a first measuring emitter ME and a second reference emitter RE, which is spaced apart from the measuring emitter as shown. Each of the two emitters is assigned an output coupler and focusing optics O1 and O1′, which are also arranged at different positions on the housing. The detector side of the housing 2 in turn comprises a measurement detector MD and a reference detector RD, which is also assigned upstream optics O2.

In one operation of the arrangement, the measuring emitter ME generates a measuring light beam MLS which it emits along an optical path in the direction of the deflecting optics O1. This directs the measuring light beam MLS out of the exit window 72 at a relatively flat angle. In this case, the term “flat angle” refers to the angle between the measuring light beam MLS and the top of the exit window 72. For its part, the reference emitter RE emits a reference light beam RLS, which is emitted from the exit window by a deflection optic O1′ at an angle that is relatively steep, in this case at a right angle. A common focal point SE for the measuring light beam MLS and the reference light beam RLS is selected so that it is located slightly above the exit window 72 and is therefore located within a sample that is placed directly on the exit window.

A measurement detector MD and a reference detector RD are provided on the detector side. The two detectors MD and RD are also spaced apart. The detectors are each preceded by an optical system O2, which collects the light entering at different angles and directs it to the corresponding detector. For this purpose, the housing comprises two entrance windows 71, which are designed in such a way that an incident light beam is guided from the optics O2 to the detector. Similar to the emitter side, it is also provided here that the light beam MGL incident on the measurement detector enters the entrance window at a different angle than a corresponding reference light beam RGL on the detector RD. In particular, the measuring light beam MGL falls onto the detector MD at a relatively flat angle, in particular at an angle similar to the angle of the measuring light beam MLS. The reference light beam RGL, on the other hand, falls essentially vertically onto the entrance window 71 and thus enters the detector RD.

Here too, a focal point is located slightly above the entry window 71 within a sample applied to the entry window. The sample thus covers both the exit window 72 and the entrance window 71, and the two focal points SE and SG are spaced apart in this embodiment.

In an operation of this arrangement with the proposed method, a measuring light beam MLS is directed at a flat angle to the point of impact SE on the sample above the exit window 72 and interacts there with particles within the sample. The measuring light beam is scattered forwards and backwards in the sample by these particles. Part of this forward-scattered light falls via the focus point SD onto the measurement detector MD, where its intensity is detected. In the same way, a reference light beam is directed onto the sample and integrates with the particles in the sample. In addition to forward scattering, this interaction also leads to a backward-scattered light component, which is detected as a backward-scattered light component RTL starting from the focal point in the reference detector RD. Due to the scattering, in particular a scattering similar to Mie scattering on particles in the order of magnitude of the wavelength of the light, there is a strong angular dependence between the forward-scattered light and the backward-scattered light component. This is recorded and set in relation to each other. Due to the dependence of the refractive index on a substance concentration in the sample, a change in concentration can be inferred from measurements that vary over time.

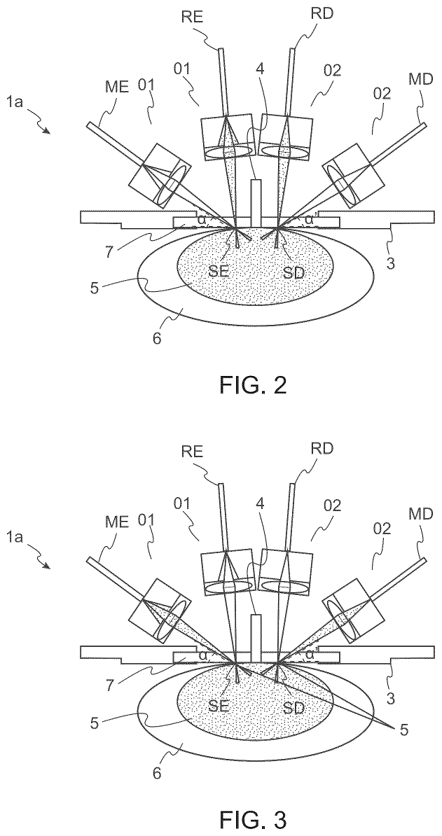

FIGS. 2 and 3 show the detector arrangement according to the proposed principle as well as the proposed non-intensive measurement method for determining a glucose concentration in a biological, in particular a human tissue. The detector arrangement 1A shown in FIG. 1 is placed on the skin surface of a person to be diagnosed. The main components of the detector arrangement are listed here again by way of example. The skin surface is marked with the reference sign 6 and is, for example, a part of the finger or another area of skin. For this purpose, the detector arrangement comprises a frame 3 with a window 7, which is placed as light-tightly as possible on the skin surface and pressed lightly against it. The seal with the skin prevents (or at least reduces) stray light from entering the measurement detector during the measurement process and thus leads to a poorer signal-to-noise ratio.

As already explained above, the detector 1A comprises a measuring emitter ME and a reference emitter RE. Optics O1 are arranged in front of the measurement emitter ME and the reference emitter RE and comprise an aperture and a downstream focusing optics. The optics O1 are designed such that they project an essentially common focal point on the skin surface 6, namely in an area SE that can be illuminated by the measuring emitter and the reference emitter RE. A common focal point is not fundamentally necessary, but is expedient in order to avoid measurement differences due to irregularities at different measurement locations.

On the receiver side, there is a measurement detector MD and a reference detector RD. These two detectors are also each preceded by a second optical system O2, which in turn comprises an aperture and one or more focusing lenses. The arrangement of the two detectors MD and RD and the upstream optics O2 is such that their respective detection point is located on the surface of the skin 6 in the area SD. Depending on the angle, light emitted from this area therefore hits the detector MD or the detector RD. The areas SE and SD are spatially separated from each other, with the distance being in the range of a few millimeters. In addition, an optical barrier 4 is provided between the emitter side and the detector side within the detector arrangement, which extends from the window 7 into the detector arrangement 1A and is intended to prevent crosstalk of light from the measuring emitter or the reference emitter to the respective detectors.

The arrangement on the emitter side with the measuring emitter ME and the reference emitter RE is designed so that a measuring light beam generated by the measuring emitter falls at the angle α onto the measuring area SE on the skin surface 6. The angle α is small and in the proposed example is in the range of about 30°, in relation to the skin surface or the surface of the window 7 placed on the skin. Consequently, the angle with respect to a normal to the skin surface in the area SE is around 60°. It should be mentioned at this point that an angle relative to the skin surface is equivalent to 90° less this angle, provided that this is then relative to the normal to the skin surface. Furthermore, it is assumed for the following examples that the exit window is parallel to the main surface of the sample. Light that emerges from the exit window at a certain angle then also falls on the skin surface at this angle.

In contrast, the reference emitter RE emits a reference light beam which falls on the area SE at a relatively steep angle of over 80° and at approximately 90° (relative to the skin surface). In other words, the reference emitter is positioned in such a way that the light it generates reaches the area SE to be illuminated essentially vertically. This arrangement also ensures that the two lines through the measurement emitter and the measurement detector virtually meet at a point below the skin or at a certain depth of the sample.

The detectors MD and RD are positioned in a similar way. The measurement detector MD is arranged so that scattered light from the area SD enters the measurement detector MD at a flat angle α′. In contrast, the reference detector RD mainly detects light that is scattered back essentially perpendicular to the surface of the skin and the surface of the window 7. In a particular embodiment, the emitters and detectors are arranged symmetrically around an axis through the optical barrier, which in turn lie along a normal to the window or skin surface. This means that the measurement emitter and the measurement detector each comprise the same angle to the normal, as do the reference emitter and the reference detector. During the measurement, the measurement detector therefore mainly receives light scattered forwards, while the reference detector mainly detects light scattered backwards. Such a symmetrical arrangement has the advantage that a later effect and, if necessary, a calibration is simplified.

Since the light beams penetrate the sample and the substance to be measured may be inside the sample and not on the surface, such an approach seems appropriate. In fact, for glucose measurements on body parts, it has been found that a certain distance between the SE and SD areas improves the measurement result. This effect can be explained on the one hand by the fact that light components reflected on the surface are effectively blocked out, so that the signal/noise ratio is improved. On the other hand, the blood for which the glucose concentration is to be determined runs at a certain depth below the skin surface.

Due to the shallow angle and the distance, the measuring light penetrates into this area and interacts with the blood in the manner described here. The light falling into the detectors has therefore traveled a longer distance and the probability of interaction with blood is greatly increased. The position of the measurement detector is again set so that the forward-scattered light is bundled in the O2 optics and fed to the detector. In this way, the detector determines the forward-scattered light from the correct depth, i.e. after it has interacted with blood.

In one operation of the proposed arrangement, a reference measurement shown in FIG. 2 is carried out first, followed by a subsequent measurement with the measuring light beam in FIG. 3. The reference light beam and the measuring light beam have the same wavelength in order to avoid a wavelength-dependent influence on the measurement. To generate the reference signal, i.e. the second light component, the reference emitter is activated and generates a reference light beam in the incident area SE. This reacts with the various particles and material components in the sample or in the skin and generates, among other things, a backscattered light in the area SD, which is collected by the optics O2 and enters the reference detector RD.

It has been shown that, in addition to the above-mentioned separation, it is also advantageous to provide the optical barrier 4 between the emitter side and the detector side in order to improve the signal-to-noise ratio. The signal measured in this way corresponds to a backscattered light component in the sample to be measured in area 5.

In a subsequent step, a measuring light beam is now generated with the measuring emitter ME and directed as a measuring light beam onto the area SE of the sample 5 of the skin 6 to be measured.

Here, too, there is interaction with various components in area 5, so that some of the incident light is scattered back in area SD and directed by the collection optics O2 onto the measurement detector MD. Due to the arrangement of the measuring emitter at a flat angle in relation to the surface of the window 7, and therefore a steep angle in relation to the normal and a correspondingly flat angle of the measuring detector MD, it is primarily forward-scattered light from the measuring light beam that is received.

As shown in FIGS. 2 and 3, the angle between the measurement light beam and the forward-scattered light is therefore significantly greater than the angle between the reference light beam from the emitter RD and the backscattered light. Due to the strong angular dependence of the scattering, a clear difference should therefore be measurable. As the angular dependence is also dependent on a change in the refractive index, the difference in the ratios at different measurement times can be used to draw conclusions about changes in the sample or to carry out a calibration.

The forward-scattered light corresponds to the first light component and, in the embodiment example, is essentially based on a scattering of the measuring light beam on red blood cells within the glucose-containing blood plasma that is comparable to Mie scattering. Here, too, it has been shown that the two measuring ranges SE and SD should be spaced apart. On the one hand, this has the advantage of reducing optical crosstalk as already mentioned; on the other hand, the separation also allows a sufficiently strong light signal to be obtained from deeper layers of the skin and in particular within the blood plasma due to forward scattering. It is assumed that the measuring light beam, which hits the skin surface at the angle α, penetrates it and reaches the bloodstream, and is then scattered in the blood plasma and the blood cells present therein.

The distance between the areas SE and SD therefore also results in a certain depth below the skin surface in which the interaction and scattering takes place. By selecting the appropriate distance between the two areas SE and SD on the skin surface, scattering can therefore be generated primarily in the area of blood plasma below the skin and the forward-scattered light can be detected by the detector MD. In addition, it has also been shown that the wavelength has a certain influence, as a slightly smaller distance is useful for generating good results with a high correlation to correct values.

The two detected signals (first and second light component, i.e. forward-scattered measurement signal and backscattered reference signal) can be set in relation to each other, whereby the resulting ratio contains a dependency of the refractive index of the blood plasma. It is assumed that the refractive index of the red blood cells is independent of a change in the concentration of glucose within the blood plasma and essentially remains constant. This means that the ratio and, in particular, the difference in the ratio between two measurements carried out at different times provides information about the change in concentration of a substance that alters the refractive index of the blood plasma.

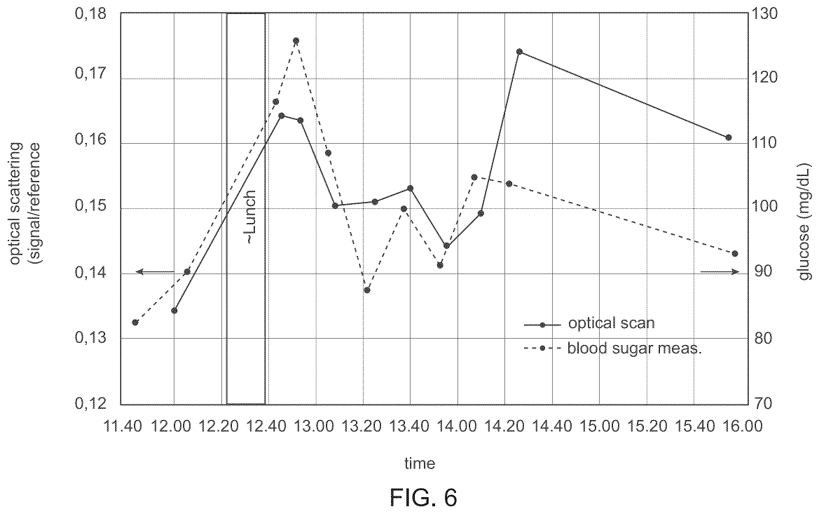

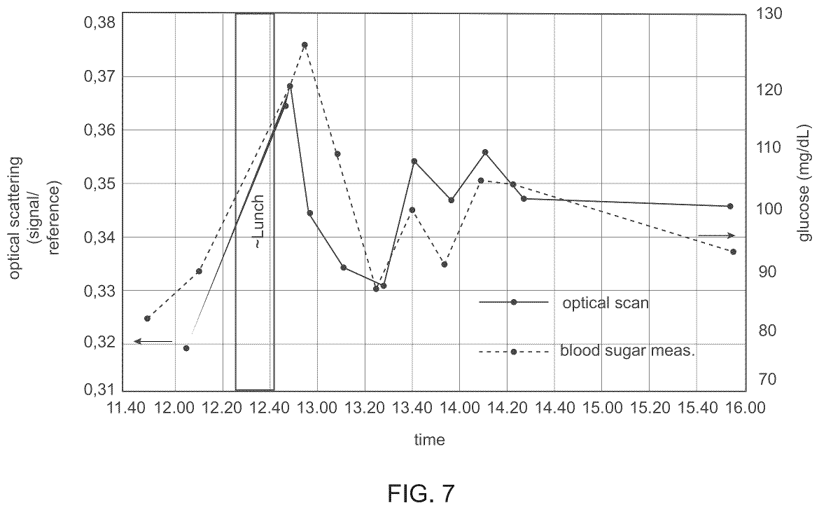

A change in the glucose concentration in the blood serves as an application example. FIGS. 6 and 7 show a comparison between the optical scan carried out with a sensor of the proposed type and an invasive method for measuring blood glucose as a reference value. FIG. 6 shows the respective time in hours on the x-axis and an optical scatter in the form of a signal/reference ratio or a corresponding glucose concentration in milligrams/deciliter on the axis. The measurement was carried out with a green measurement and reference light beam, whereby the distance between the SD and MD areas was approximately 2 mm.

As can be seen from the curve of the blood glucose measurement, the glucose concentration rises steeply during the increase in food at around 12:30 to 12:40 and then falls again slightly with some fluctuations. The optical scan shows a very similar course, which suggests a correlation between a change in the refractive index in the blood plasma during this period. The optical scan can be easily calibrated by comparison with the blood glucose measurement.

The difference and variations between the invasive blood glucose measurement and the optical scanning according to the proposed principle can be explained by the color of the scattered light (in this case with a wavelength in the green range), the shape and surface texture of the finger and other parameters. In addition, this measurement was carried out at a small distance of only 2 mm between the area of the scattered light SE and the area of the backscattered or forward scattered light SD, whereby the wavelength used here shows a good correlation.

FIG. 7 shows a second measurement at a different wavelength (near infrared range) and also a greater distance between the SE and SD areas on the surface of the skin of around 4 mm. Here, an even greater correspondence of the time course between the optical scan on one side and the invasive blood glucose measurement on the other side can be seen. The invasive blood glucose measurement given by the glucose value in milligrams per deciliter is the same between FIGS. 6 and 7, only the strength of the optical scattering, i.e. the ratio of measurement signal to reference signal, is different for the two measurements performed due to the different wavelength of the different distance. However, the high correlation between the optical scanning and the invasive blood glucose measurement indicates good agreement and a clear measurable dependency.

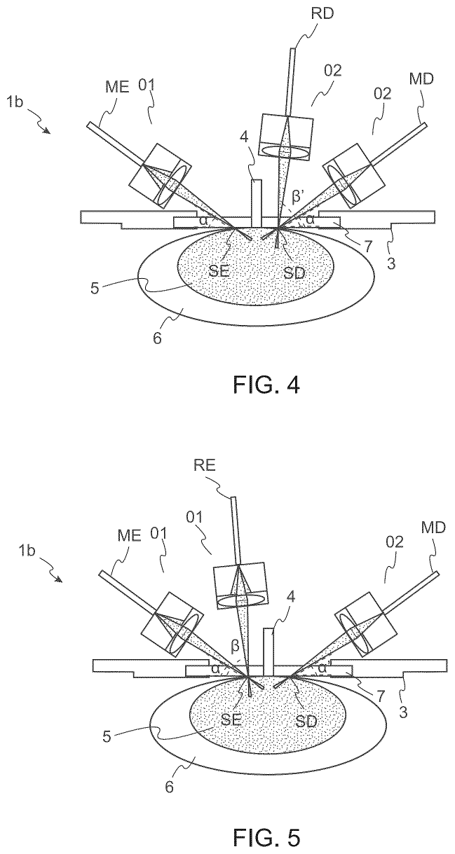

FIGS. 4 and 5 show alternative embodiments of a detector that is suitable for carrying out the proposed method. According to the basic principle, the arrangements allow light components scattered at different angles to be evaluated and related to each other. FIG. 5 shows an arrangement 1b that comprises only one measuring emitter ME but two detectors RD and MD. Similar to the embodiment in FIG. 3, the measuring emitter ME is arranged in such a way that the light emitted by it falls on the area of the sample in the area SE at a flat angle α to the surface of the window 7 of the detector. In other words, the light beam emitted by the emitter ME strikes the surface of the sample at a large angle with respect to the normal.

On the detector side, the reference detector RD is positioned so that it receives the light scattered and emitted essentially perpendicular to the sample. In contrast, the measurement detector MD is positioned so that it only detects forward-scattered light, i.e. light at a flat angle α, relative to the surface of the window 7. Similar to the previous embodiments, the detector RD is thus designed to detect backscattered light and the measurement detector MD to detect forward-scattered light, in particular caused by scattering comparable or similar to Mie scattering.

In one operation of the detector arrangement 1b, a light beam is generated by the emitter ME and directed onto the area SE of the sample. The light beam can serve both as a measurement beam and as a reference light beam. The incident light interacts with the sample and leads to a forward-scattered light component in the area SD as well as a second and a different angle backscattered light component. The second light component is therefore also referred to as backscattered light and reaches the reference detector RD via the collection optics O2. The forward-scattered light is collected and detected via a second collection optic in front of the measurement detector MD.

In this embodiment, it is possible to generate a single light beam and have it interact with the sample. Both a detection of backward-scattered light using the reference detector RD and forward-scattered light using the measurement detector MD can be received simultaneously. Alternatively, it is possible to emit the light beam as a pulsed light beam in order to receive the backward-scattered light component with detector RD and the forward-scattered light component with detector MD in two different measurements.

FIG. 6 shows an alternative design that makes use of the fact that it is essentially the angular difference between the incident light and the portion of light scattered in the sample that is important. It is therefore also possible to dispense with one of the two detectors, but instead provide differently positioned emitters. This means that the first light component (measurement signal) and the second light component (reference signal) are detected with the same detector. Furthermore, an arrangement 1b comprises two different emitters MD and RD for generating a measurement light beam and a reference light beam. The two emitters are positioned in such a way that they direct the light they emit at different angles α and β onto the area SE. Specifically, the light from the measuring emitter falls at a flat angle α onto the area SE (again in relation to the horizontal surface of the window 7, while the second light beam from the reference emitter falls essentially vertically, i.e. at a very steep angle, onto the area SE.

The measurement detector MD, on the other hand, is arranged at an angle α′ so that it receives the light scattered by the area SD at this angle. This arrangement means that a reference light signal that falls on the area SE and generates backscattered light in the area SD always comprises a different total angle than the measurement light beam that falls on the area SE at the flatter angle α. In the example, the angle between the measurement light beam and the scattered light on the detector MD is greater than the angle of the reference light beam and the scattered light.

The method proposed here makes use of the fact that the angular dependence manifests itself in different ways. On the one hand, this can result directly from the position of the reference or measurement detector and the two emitters. In the example shown in FIG. 6, the angle dependence is largely determined by the angle between the incident light and the backscattered light. In the case of the reference light beam, i.e. the light beam emitted by the emitter RE, the angle between the emitter and the detected light is significantly smaller than in the case of detection of forward-scattered light, which is generated by the measuring light beam from measuring emitter ME. Accordingly, there is also an angle dependence here, the ratio of which changes depending on the glucose concentration within the sample 5.

This law also applies in the same way to the other examples. In all of them, the angle between the reference light directed at the sample and the backscattered and detected light is smaller than the angle between the measuring light beam directed at the sample and the detected forward-scattered light. This is necessary because the forward-scattered light and the backscattered light result from scattering similar to Mie scattering. The formation of a ratio is robust against skin changes or other parameters that change the measurement and allows conclusions to be drawn about the glucose concentration.

In addition, the method also allows conclusions to be drawn about other health-relevant parameters. Since the scattering within a certain tissue volume depends on the concentration of the scattering objects, the proposed method also allows the amount of water in relation to the amount of red blood cells to be determined. This provides an indication of possible hydration or dehydration.

### REFERENCE LIST

1a detector device
3 frame
4 optical barrier
5 area of interaction
6 sample area
7 window
ME measurement emitter
RE reference emitter
RD reference detector
MD measurement detector
O1 emitter optics
O2 detector optics

1. A method for determining a substance concentration in a particle-containing liquid, in particular of glucose in blood, wherein a refractive index of the liquid is dependent on a concentration of the substance dissolved therein, comprising the steps:
emitting a measuring light beam of at least one wavelength onto a sample containing the liquid;
detecting a first light component scattered by scattering on the particles contained in the liquid at a first angle, which corresponds in particular to a forward scattering of the light component;
detecting a second light component scattered at a second angle by the particles contained in the liquid;
determining a concentration or a proportion of the substance in the liquid from the detected first and second light components, in particular by forming a ratio from the detected first and second light components.

2. The method according to claim 1, in which an angle between the first light component and the measuring light beam is at least 60° and in particular at least 90° greater than an angle between the second light component and a light beam producing this light component on the sample.

3. The method according to claim 1, in which the first angle relative to a normal to a surface of the sample is greater than 45° and in particular greater than 55°; and/or in which the second angle relative to a normal to a surface of the sample is less than 350 and in particular less than 20°.

4. The method according to claim 1, in which the measuring light beam is irradiated at a third angle with respect to a normal to a surface of the sample, which angle is greater than 45° and in particular greater than 60°.

5. The method according to claim 1, in which an angle between the measuring light beam and the first light component generated by scattering on the particles contained in the liquid is more than 900 and in particular more than 120°.

6. The method according to claim 1, in which an angle between the measuring light beam and the scattered second light component is less than 60° and in particular less than 30°.

7. The method according to claim 1, wherein the step of emitting a measuring light beam comprises emitting a measuring light beam of a first wavelength and emitting a measuring light beam of a second wavelength.

8. The method according to claim 1, wherein the step of detecting a second light component scattered by the particles contained in the liquid comprises:
emitting a reference light beam of at least one wavelength onto the sample;
detecting of light scattered by the reference light beam as a second light component at the second angle;

9. The method according to claim 8, in which the reference light beam is irradiated at a fourth angle with respect to a normal to a surface of the sample, which angle is less than 45° and in particular less than 30°.

10. The method according to claim 8, wherein the measurement light beam and the reference light beam are emitted at different times.

11. The method according to claim 8, wherein the reference light beam and the measurement light beam comprise the same wavelengths or different wavelengths.

12. The method according to claim 1, in which a point of incidence of the measuring light beam on the sample is spaced apart from a point from which the first and/or second light beam is detected.

13. The method according to claim 12, in which the distance is in the range from 1 mm to 8 mm and in particular between 2 mm and 4 mm.

14. An optoelectronic sensor for detecting a concentration of a substance in a sample, comprising:
a housing with an outlet window and an optional inlet window,
at least one emitter unit, which is arranged below the exit window and is designed to emit light at at least a first angle from the exit window onto the sample;
at least one photodetector unit, which is arranged below the entrance window and is designed to detect light scattered by the sample and incident at at least a first angle;
wherein the sensor is adapted to detect a first light component in response to a first emitted light and a second light component in response to a second emitted light; wherein an angle between the first light component and the first emitted light is at least 60° and in particular at least 90° greater than an angle between the second light component and the second emitted light;
an evaluation unit which is coupled to the at least one photodetector unit and is designed to determine a substance concentration in the sample from a ratio of the detected first and second light components.

15. The optoelectronic sensor according to claim 14, in which first and second light components are at least partially formed by scattering on scattering particles in the sample, wherein in particular the first light component is formed by forward-scattered light and the second light component is formed by backward-scattered light.

16. The optoelectronic sensor according to claim 14, wherein the at least one emitter unit comprises:
a measuring emitter designed to emit light at a first predetermined angle from the exit window onto the sample;
optionally a reference emitter, which is designed to emit light at a second predetermined angle from the exit window onto the sample;
wherein the second predetermined angle relative to a normal to the sample surface is smaller than the first predetermined angle.

17. The optoelectronic sensor according to claim 16, wherein the first predetermined angle relative to a normal to a surface of the sample is greater than 45° and in particular greater than 55°; and/or wherein the second predetermined angle relative to a normal to a surface of the sample is less than 350 and in particular less than 20°.

18. The optoelectronic sensor according to claim 14, comprising the at least one photodetector unit:
a measurement detector designed to detect light scattered by the sample and incident at at least a first predetermined angle;
optionally a reference detector adapted to detect light scattered by the sample and incident at at least a second predetermined angle; wherein the second predetermined angle is smaller than the first predetermined angle with respect to a normal to the sample surface.

19. The optoelectronic sensor according to claim 18, in which the first predetermined angle relative to a normal to a surface of the sample is greater than 45° and in particular greater than 55°; and/or in which the second predetermined angle relative to a normal to a surface of the sample is less than 350 and in particular less than 20°.

20. The optoelectronic sensor according to claim 14, further comprising an optical barrier arranged in the housing and extending from the exit window towards the bottom of the housing between the emitter unit and the photodetector unit.

21. The optoelectronic sensor according to claim 14, wherein the emitter unit and/or the photodetector unit comprises an optical system configured to generate a focal spot on the sample.

22. The optoelectronic sensor according to claim 14, wherein focus point on the sample generated by light from the emitter unit is spaced from a point from which outgoing light is detectable at the at least one first angle by the photodetector unit.

23. The optoelectronic sensor according to claim 21, wherein the distance between the focal point and the point is in the range of 1 mm and 6 mm and in particular in the range of 2 mm and 4 mm.
